(19) United States
(12) Patent Application Publication
(10) Pub. No.: US 2024/0023899 A1
(43) Pub. Date: Jan. 25, 2024

(54) APPARATUS AND METHOD FOR RECONSTRUCTING HIGH-FREQUENCY BIO-SIGNAL BASED ON NEURAL NETWORK MODEL

(71) Applicant: DAEGU GYEONGBUK INSTITUTE OF SCIENCE AND TECHNOLOGY, Daegu (KR)

(72) Inventors: Hongki KANG, Daejeon (KR); Kyong Hwan Jin, Daegu (KR); Jaeha KUNG, Daegu (KR); Jaewon LEE, Daegu (KR); Junghyup LEE, Daegu (KR); Nari HONG, Daegu (KR)

(21) Appl. No.: 18/207,316

(22) Filed: Jun. 8, 2023

(30) Foreign Application Priority Data
Jul. 22, 2022 (KR) 10-2022-0091283

(51) Int. Cl. A61B 5/00; A61B 5/11

(52) U.S. Cl. CPC A61B 5/7278; A61B 5/1126; A61B 5/7267; A61B 5/37

(57) ABSTRACT

A method of restoring a high-frequency biosignal includes the steps of loading a first biosignal by a processor and converting the first biosignal that is a low-frequency signal into a second biosignal that is a high-frequency signal on the basis of a first neural network model.

### BACKGROUND OF THE INVENTION

### Field of the Invention

The present disclosure relates to a method and apparatus for restoring a high-frequency biosignal from a low-frequency biosignal.

### Description of the Related Art

There are conventional techniques for measuring and analyzing various types of biosignals including brain signals. For example, an electroencephalogram (EEG) and an electrocardiogram (ECG) are representative biosignals used for disease determination and the like.

Brain signals are classified into various types of brain signals according to measurement locations, including electroencephalograms. For example, referring to FIG. 1A, various brain signals such as LFP, MUA, and SUA classified according to signal processing methods performed on an electroencephalogram 1010 measured mainly from the scalp, an ECoG 1020 measured on or below the dura surrounding the brain, and a spike-based brain signal 1030 measured by inserting a needle-shaped electrode into the brain, can be measured. Thereamong, the ECoG 1020 and the spike-based brain signal 1030 can be measured by an invasive method that requires a surgical method such as scalp or skull cutting, and the EEG 1010 can be measured by a non-invasive method of placing electrodes on the scalp. Therefore, there are cases in which a brain signal needs to be measured by an invasive method depending on a disease or a brain signal to be analyzed from a subject.

In addition, biosignals are composed of components of different frequency bands according to the signals. For example, referring to the results of FIG. 1B measured in Prior Art 1, it can be ascertained that the above-described LFP, ECoG, and EEG are composed of components of different frequency bands. Referring to the results of FIG. 10 measured in Prior Art 2, it can be ascertained that ECoG/LFP are signals of a higher frequency band than EEG, and that the spike-based signal is composed of components of a higher frequency band than ECoG/LFP. That is, in the case of an amplitude measured based on the voltage of an electrode, it can be ascertained that intra-brain LFP and ECoG signals have higher frequency components than a scalp EEG, and the spike-based signal has higher frequency components.

In addition, there are increasing cases of implementing conventional sensors for measuring biosignals for monitoring subjects as wearable sensors. However, in the case of a wearable biosignal measurement device, it is very important to reduce power consumption and secure a data storage space because biosignals of a subject need to be measured, stored, and transmitted for a long time.

Conventionally, there is an adaptive sampling method for selectively recording data of high importance at the time of measuring biosignals from a subject (Prior Art 3). Prior Art 2 utilizes a method of irregular sampling focusing on important events. However, in Prior Art 2, there is a risk of losing important data in a process of recording data of a selective time range rather than data of the entire measurement time range, and at the time of restoring signals in a restoration system using recorded signals, irregular sampling timing information is essential.

### CITED REFERENCE

### Non-Patent Document

Prior Art 1: “The origin of extracellular fields and currents—EEG, ECoG, LFP and spikes,” Nature Reviews Neuroscience, 2012 June, Vol. 13, No. 6

Prior Art 2: “A Low-Power ECoG/EEG Processing IC with Integrated Multiband Energy Extractor,” IEEE TRANSACTIONS ON CIRCUITS AND SYSTEMS, 2011 September, Vol. 58, No. 9

Prior Art 3: “A neural algorithm for the non-uniform and adaptive sampling of biomedical data,” Computers in Biology and Medicine, Apr. 1, 2016, Volume 71

### SUMMARY OF THE INVENTION

There is a need for a method of obtaining biosignals such as ECoG and spike-based brain signals without using conventional invasive biosignal measurement methods.

In addition, there is a need for a method of solving problems regarding power and storage spaces of wearable biosignal measurement devices.

Embodiments of the present disclosure provide a method and apparatus to solve such problems.

An embodiment of the present disclosure provides an apparatus and method for restoring a biosignal of a high frequency band from a biosignal of a low frequency band.

An embodiment of the present disclosure provides an apparatus and method for restoring a high-frequency biosignal sampled at a high rate (high-sampled) from a low-frequency biosignal sampled at a low rate (low-sampled).

An embodiment of the present disclosure provides an apparatus and method for restoring a biosignal to be measured in an invasive way from a noninvasively acquired biosignal.

An embodiment of the present disclosure provides an apparatus and method for training a learning model based on a neural network model that restores a biosignal of a high frequency band from a biosignal of a low frequency band.

In accordance with an aspect of the present disclosure, the above and other objects can be accomplished by the provision of a method of restoring a high-frequency biosignal, including loading a first biosignal by a processor, and converting, by the processor, the first biosignal corresponding to a low-frequency signal into a second biosignal corresponding to a high-frequency signal on the basis of a first neural network model.

In accordance with another aspect of the present disclosure, there is provided an apparatus for restoring a high-frequency biosignal, including a processor and a memory electrically connected to the processor and storing at least one code executed by the processor, wherein the memory stores code for causing, when executed by the processor, the processor to convert a first biosignal corresponding to a low-frequency signal into a second biosignal corresponding to a high-frequency signal on the basis of a first neural network model.

### BRIEF DESCRIPTION OF THE DRAWINGS

The above and other objects, features and other advantages of the present disclosure will be more clearly understood from the following detailed description taken in conjunction with the accompanying drawings, in which:

FIGS. 1A, 1B and 1C are diagrams illustrating conventional brain signal measurement locations and frequency bands of various types of brain signals;

FIG. 2 is a diagram illustrating an environment in which a method of restoring a high-frequency biosignal is performed or an apparatus for restoring a high-frequency biosignal is operated according to an embodiment of the present disclosure;

FIG. 3 is a block diagram showing a configuration of a high-frequency biosignal restoration apparatus according to an embodiment of the present disclosure;

FIG. 4 is a block diagram showing a configuration of a sensor device for measuring a low-sampled and low-frequency biosignal according to an embodiment of the present disclosure;

FIG. 5 is a flowchart illustrating a method of restoring a high-sampled and/or high-frequency biosignal.

FIGS. 6 and 7 are diagrams illustrating a method of training a neural network model for restoring high-frequency biosignal and a structure of the neural network model according to embodiments of the present disclosure;

FIGS. 8A and 8B are diagram illustrating a ground truth (GT) signal of a neural network model for restoring a high-frequency biosignal and a method of generating the GT signal according to an embodiment of the present disclosure;

FIGS. 9, 10A and 10B show high-frequency biosignal restoration results according to embodiments of the present disclosure.

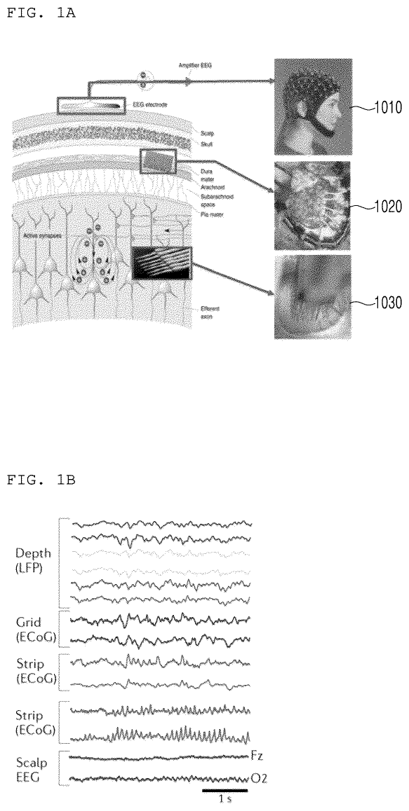

### DETAILED DESCRIPTION OF THE INVENTION

Hereinafter, embodiments disclosed in the present specification will be described in detail with reference to the accompanying drawings, but the same or similar components are denoted by the same reference numerals and redundant descriptions thereof will be omitted. The suffixes “module” and “part” of elements herein are used for convenience of description and thus can be used interchangeably and do not have any distinguishable meanings or functions. In the following description of the embodiments disclosed in the present specification, a detailed description of known functions and configurations incorporated herein will be omitted when it may obscure the subject matter of the present disclosure. In addition, the accompanying drawings are provided only for ease of understanding of the embodiments disclosed in the present specification, do not limit the technical spirit disclosed herein, and include all changes, equivalents and substitutes included in the spirit and scope of the present disclosure.

The terms “first” and/or “second” are used to describe various components, but such components are not limited by these terms. The terms are used to discriminate one component from another component.

When a component is “coupled” or “connected” to another component, it should be understood that a third component may be present between the two components although the component may be directly coupled or connected to the other component. When a component is “directly coupled” or “directly connected” to another component, it should be understood that no element is present between the two components.

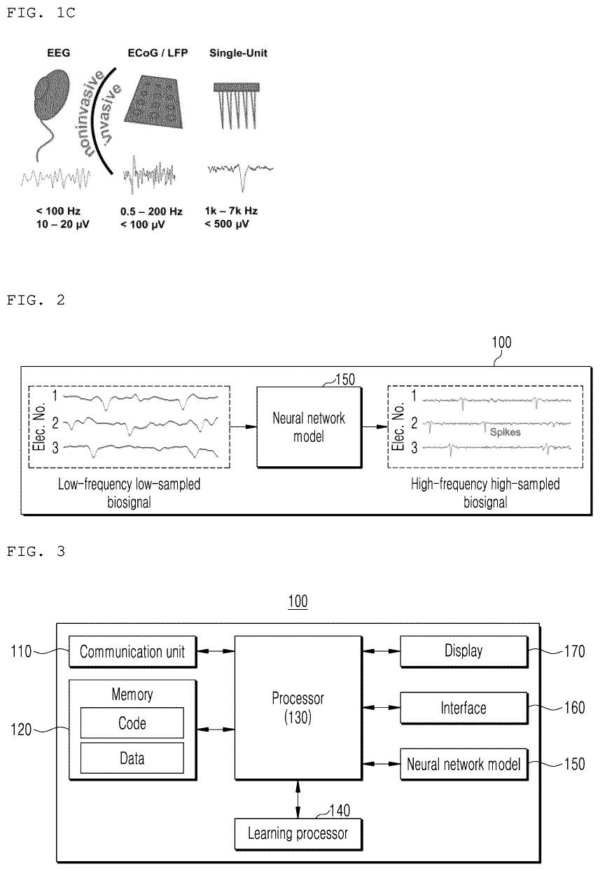

An environment in which a method of restoring a high-frequency biosignal is performed or an apparatus for restoring a high-frequency biosignal is operated according to an embodiment of the present disclosure will be described with reference to FIG. 2.

The method of restoring a high-frequency biosignal according to an embodiment of the present disclosure may be performed in a biosignal reception device or a biosignal processing device. In this specification, a high-frequency biosignal restoration apparatus 100 (hereinafter referred to as a restoration apparatus) will be described as including a biosignal reception device or a biosignal processing device.

The biosignal reception device may be a device that periodically or non-periodically receives measured biosignals through wired/wireless communication from a wearable biosignal measurement sensor device as shown in FIG. 4 that is attached to the body of a subject to measure electrical signals (e.g., signals representing changes in the amplitudes of electrode signals over time measured from electrodes attached to the scalp, skin, etc. of the subject) from the subject.

When the high-frequency biosignal restoration method according to an embodiment of the present disclosure is performed in the biosignal reception device, an output obtained by pre-processing a received low-sampled and/or low-frequency biosignal and then inputting the pre-processed biosignal to a learning model 150 trained to restore a high-sampled and/or high-frequency biosignal from a low-sampled and/or low-frequency biosignal may be restored as a final bio signal.

A biosignal processing device is a computing device that processes biosignals and may include any device capable of driving the learning model 150 based on a neural network model. For example, server devices, workstations, laptops, personal computers, tablet computers, and the like may receive biosignals on the basis of a network or other interfaces and input the received biosignals into a learning model distributed by a learning model distribution device to restore high-sampled and/or high-frequency biosignals.

The learning model 150 trained to restore a high-sampled and/or high-frequency biosignal from a low-sampled and/or low-frequency biosignal may be a deep-learning-based learning model based on a neural network model and including a plurality of convolutional layers. The learning model 150 may be trained in a separate learning model distribution device, or may be trained in the restoration apparatus 100 in an embodiment.

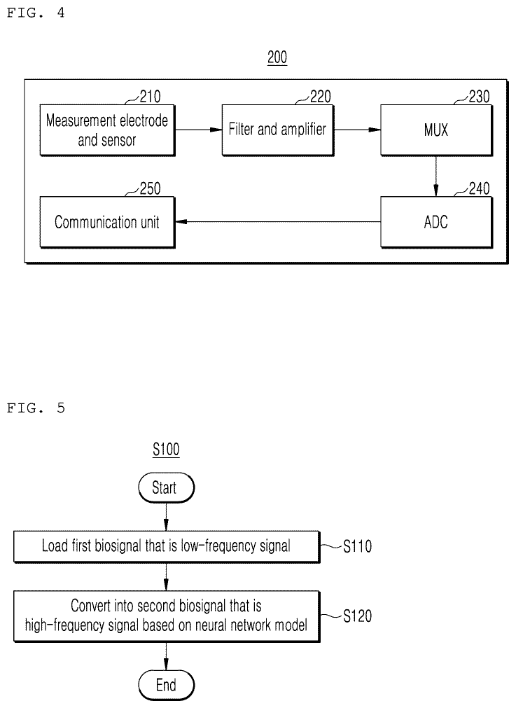

A configuration of the restoration apparatus 100 for restoring a high-frequency biosignal according to an embodiment of the present disclosure will be described with reference to FIG. 3.

The restoration apparatus 100 may include a communication unit 110 that receives a learning model from a learning model distribution device or receives biosignals, a memory 120 that loads and temporarily stores code for driving a processor 130, low-sampled and/or low-frequency biosignals, and a learning model, the processor 130 that controls components and perform operations, a learning processor 140 that accelerates and processes deep learning-based neural network operations, the learning model trained to restore a high-sampled and/or high-frequency bio-logical from a low-sampled and/or low-frequency biosignal, an interface 160 that receives instructions from a user and provides results, and a display 180 that displays various results. The learning processor 140 may be a processor that is based on a GPU or supports dedicated AI acceleration.

Hereinafter, each component of the restoration apparatus 100 will be described in detail.

In one embodiment, the restoration apparatus 100 may include the communication unit 110 for performing communication with a user terminal or a learning model distribution device or receiving low-sampled and/or low-frequency biosignals from a wearable biosignal measurement sensor device 200.

The communication unit 110 may include a wireless communication unit or a wired communication unit.

The wireless communication unit may include at least one of a mobile communication module, a wireless Internet module, a short-range communication module, and a location information module.

The mobile communication module transmits/receives radio signals to/from at least one of a base station, an external terminal, and a server through a mobile communication network constructed according to Long Term Evolution (LTE), which is a communication protocol for mobile communication.

The wireless Internet module is a module for wireless Internet access and may be provided inside or outside the restoration apparatus 100 and use wireless LAN (WLAN), Wi-Fi, Wi-Fi Direct, Digital Living Network Alliance (DLNA), and the like.

The short-range communication module is a module for transmitting and receiving data through short-range communication, and may use Bluetooth™, radio frequency identification (RFID), infrared data association (IrDA), ultra-wideband (UWB), ZigBee, near field communication (NFC), and the like.

The location information module is a module for obtaining the location of the restoration apparatus 100 and may be a global positioning system (GPS) module based on satellite navigation technology or a module for obtaining a location on the basis of wireless communication with a wireless communication base station or a wireless access point. The location information module may include a Wi-Fi module.

In one embodiment, the restoration apparatus 100 may include an input unit or an output unit as the interface 160 for user input.

The input unit may include a user interface (UI) including a microphone and a touch interface for receiving information from a user, and the user interface may include a mouse and a keyboard as well as mechanical and electronic interfaces implemented in the apparatus, and the operation method and form thereof are not particularly limited as long as it can receive instructions from a user. Electronic interfaces include a display capable of touch input.

The output unit displays output of the restoration apparatus 100 to deliver information to the user, and may include the display 180, LEDs, a speaker, and the like for displaying visual, auditory, or tactile output.

The restoration apparatus 100 may include a peripheral device interface for data transmission to various types of connected external devices, a memory card port, an external device input/output (I/O) port, etc.

The learning model 150 based on a neural network model may include a CNN, a region-based CNN (R-CNN), a convolutional recursive neural network (C-RNN), a fast R-CNN, a faster R-CNN, a region-based fully convolutional network (FCN), You Only Look Once (YOLO) or a neural network in a single shot multibox detector (SSD) structure.

In one embodiment, the learning model 150 may include a plurality of convolutional layers, and may include an Image Restoration using Swin Transformer (SwinIR) block modified and/or trained to fit one-dimensional biosignals. The SwinIR block may include a plurality of residual Swin transformer blocks (RSTBs), and each RSTB may include a plurality of Swin transformer layers (STLs). SwinIR modified and/or trained to suit the embodiments of the present disclosure will be described below with reference to FIGS. 6 and 7.

The learning model 150 may be implemented by hardware, software, or a combination of hardware and software, and when part or all of the learning model is implemented as software, one or more instructions constituting the learning model may be stored in a memory.

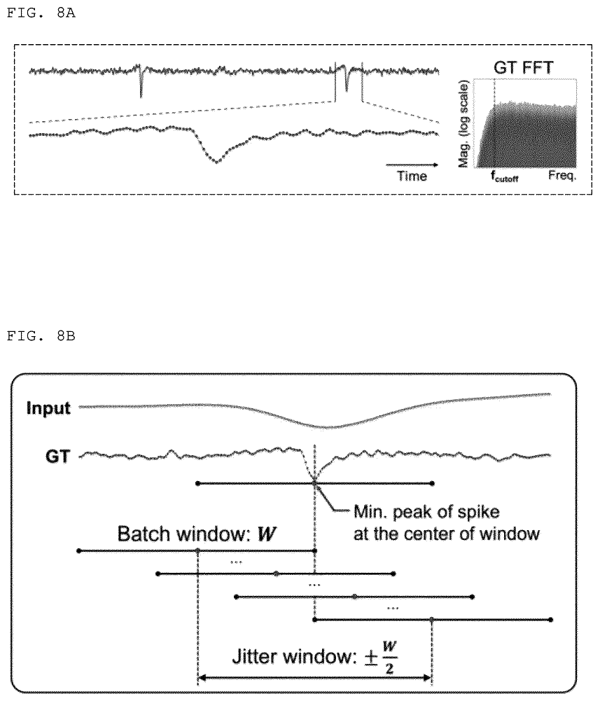

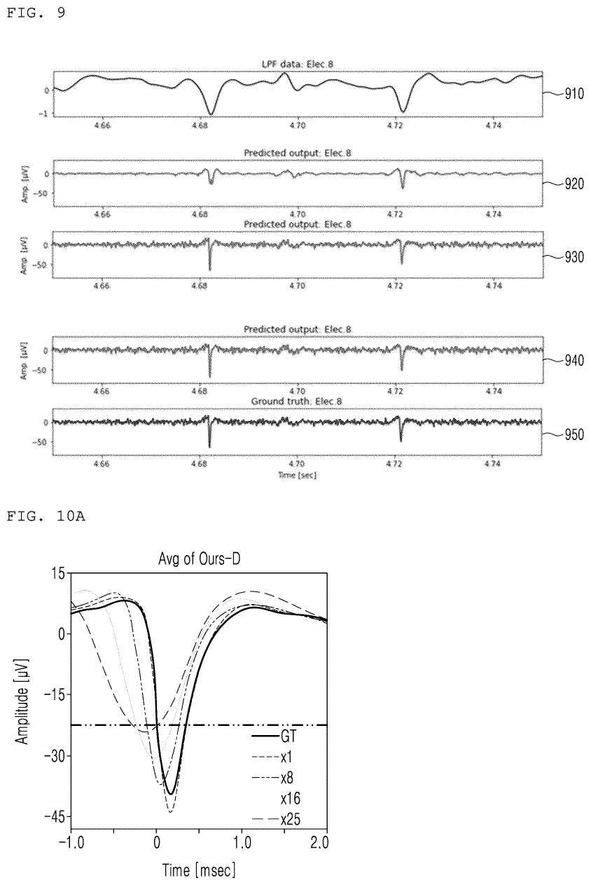

The learning model 150 is a learning model trained to restore a high-sampled and/or high-frequency biosignal by receiving a low-sampled and/or low-frequency biosignal as an input. Training data for training the learning model 150 will be described below with reference to FIGS. 8A and 8B.

A configuration of the wearable biosignal measurement sensor device 200 according to an embodiment of the present disclosure will be described with reference to FIG. 4.

The sensor device 200 may include an electrode 210 attached to the body of a subject to obtain an electrical signal from the subject, a filter and an amplifier 220 for reducing noise of a low frequency band in the electrical signal obtained from the electrode and amplifying the signal with reduced noise. The amplifier 220 may have an instrumentation amplifier structure using an OP-Amp. Various filter and amplifier structures used in conventional biosignal measurement devices may be used. When the electrode 210 is composed of a plurality of channels, the sensor device 200 may include a multiplexer 230 for selecting a signal from each channel, an ADC 240 for sampling an amplified signal to convert the amplified signal into a digital signal, and a communication unit 250 that transmits a low-sampled and/or low-frequency biosignal converted into a digital signal.

In one embodiment, the filter and amplifier 220, the multiplexer 230, the ADC 240, and/or the communication unit 250 may be integrated into one chip and implemented as an analog signal processor.

The communication unit 250 may use a WLAN, Wi-Fi, or Wi-Fi Direct, or short-range communication such as Bluetooth, infrared communication, UWB, ZigBee, and the like.

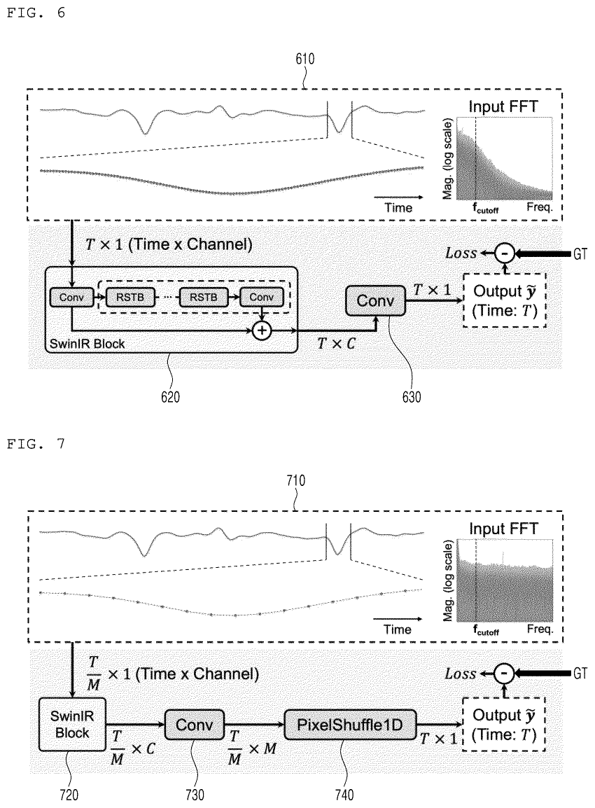

A method of restoring a high-sampled and/or high-frequency biosignal in the restoration apparatus 100 will be described with reference to FIG. 5.

The restoration apparatus 100 may receive a low-sampled and/or low-frequency first biosignal from a biosignal sensor device. Alternatively, the restoration apparatus 100 may receive the first biosignal through a network or other interfaces using a separate device for restoration of a high-frequency biosignal. The restoration apparatus 100 loads the received first biosignal into the memory (S110).

The first biosignal may be a biosignal composed of a plurality of channel signals measured at the same time from the same subject. In this case, a biosignal for each channel may be input to a neural network model.

The restoration apparatus 100 restores a high-sampled and/or high-frequency second biosignal by inputting the first biosignal to a learning model based on a neural network model (S120). The learning model of the restoration apparatus 100 may transform the first biosignal into the second biosignal by restoring the second biosignal from a feature map generated from the first biosignal through a learning model including a plurality of convolutional layers.

A learning model based on a neural network model will be described with reference to FIGS. 6 and 7.

In another embodiment, referring to FIG. 6, a high-sampled and low-frequency first biosignal 610 obtained by upsampling a low-sampled low-frequency biosignal may be input to SwinIR block 620, which is a first neural network model based on a plurality of RSTBs. Each RSTB may include a plurality of STLs.

The high-sampled and low-frequency first biosignal 610 may be a one-dimensional biosignal measured over a time length T.

The first biosignal 610 may be input to the SwinIR block 620, which is a first neural network model based on a plurality of RSTBs. Each RSTB may include a plurality of STLs. Each STL may include multi-head self-attention (MSA), and each RSTB may be composed of repetitions of an STL.

The SwinIR block 620 may have a structure in which an output of a convolutional layer, which is an input layer, is skip-connected to an output of another convolutional layer of an output stage.

In one embodiment, the number of RSTBs, the number of STLs, the number of feature map channels, and the kernel size of a one-dimensional convolutional layer of the SwinIR block 620 may be 6, 6, 180, and 3, respectively.

The SwinIR block 620 may output a plurality of channel signals (T×C, feature map channels) having the same time length T as the first biosignal 610 and pass the channel signals through a separate one-dimensional convolutional layer 630 to convert the channel signals into a one-dimensional signal. The signal output from the one-dimensional convolutional layer 630 may be a high-sampled and high-frequency second biosignal.

In a training process, a learning model including the first neural network model 620 may be trained on the basis of an output of a loss function having, as an input, a difference between a ground truth (GT) signal labeled on the input signal 610 that is training data and an output signal from the learning model.

In another embodiment, referring to FIG. 7, a low-sampled and low-frequency first biosignal 710 may be a signal sampled at a lower sampling rate M based on the same time length T than the first biosignal 610 of FIG. 6.

The first biosignal 710 may be input to a SwinIR block 720, which is a first neural network model based on a plurality of RSTBs. Each RSTB may include a plurality of STLs. The SwinIR block 720 of FIG. 7 may have the same structure as the SwinIR block 620 of FIG. 6 although the size of some convolutional layers may be different.

The SwinIR block 720 may generate a plurality of channel signals (T/M×C, feature map channels) having the same time length as the first biosignal 710 with C channels, and pass the channel signals through a separate one-dimensional convolutional layer 730 to convert the channel signals into a one-dimensional signal. Thereafter, the one-dimensional signal that has passed through the one-dimensional convolutional layer 730 is input to a second neural network model 740 that improves a sampling frequency, and a signal output from the second neural network model 740 may be a high-sampled and high-frequency second biosignal.

In an embodiment, the second neural network model 740 that improves a sampling frequency may be a learning model based on an efficient sub-pixel convolutional neural network (ESPCN).

In a training process, the learning model including the first neural network model 720 and the second neural network model 740 may be trained on the basis of an output of a loss function having, as an input, a difference between a GT signal labeled on the input signal 710 that is training data and an output signal from the learning model.

In one embodiment, in the neural network model according to the embodiments of the present disclosure, an input biosignal and an output biosignal may have different frequency bands.

For example, the neural network model may receive a first biosignal in a low frequency band and generate a second biosignal in a high frequency band.

In addition, the neural network model may receive a non-invasively measured biosignal (e.g., EEG signal) and generate an invasively measured biosignal (e.g., ECoG or spike-based brain signal). That is, input signals may be converted into different types of biosignals obtained from different parts of the human body. Therefore, it is possible to restore invasively measured biosignals without surgery from the output of a non-invasive biosignal sensor device.

Furthermore, one type of invasively measured biosignal (e.g., ECoG) may be received and another type of invasively measured biosignal (e.g., spike-based brain signal) may be generated. Accordingly, it is possible to restore an invasively measured biosignal that requires more dangerous or major surgery from the output of a less invasive biosignal sensor device.

In addition, the neural network model may restore different types of high-frequency biosignals generated by activities of the same body organ from low-frequency biosignals. For example, an electrocardiogram (ECG) signal in a high frequency band may be generated from a plethysmograph (PPG) in a low frequency band. Further, a movement of a muscle may be measured by a motion sensor based on an acceleration and/or a gyro and converted into an electromyography (EMG) signal, for example.

A training method for training a learning model including a neural network model that restores a high-sampled and/or high-frequency biosignal from a low-sampled and/or low-frequency biosignal and a method of constructing training data will be described with reference to FIGS. 8A and 8B. In one embodiment, the neural network model may include a SwinIR block and may be trained based on a fixed training rate of le-4 on the basis of a mean square error (MSE) loss function and an Adam optimizer, but the present disclosure is not limited thereto.

Training data may be obtained on the basis of training data in which input signals, which are low-frequency signals, are labeled with ground truth (GT) signals, which are high-frequency signals.

In an embodiment, input signals and GT signals may be EEG/ECoG, EEG/spike-based signal, ECoG/spike-based signal, PPG/ECG, and motion sensor signal/EMG. Input signals and GT signals can be measured at different body parts of the same subject at the same time.

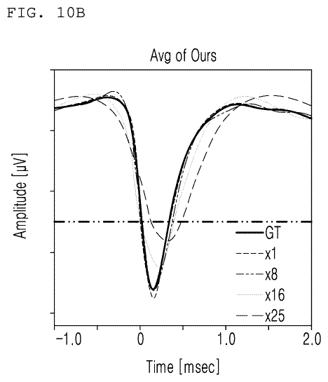

In one embodiment, input signals may be obtained by sampling signals measured from cultured cells through a microelectrode array (MEA) at a low sampling frequency through a low pass filter, and GT signals may be obtained by sampling the signals at a high sampling frequency. FIGS. 9 to 10 show experimental results of the learning model trained with such training data.

In an embodiment, GT signals of a preset ratio of first training data in each mini-batches of the training data may be configured to include at least one spike. Accordingly, it is possible to accurately restore a high-frequency signal including a spike from a low-frequency biosignal.

For example, when a GT signal is as shown in FIG. 8A, a low-frequency signal corresponding to the time at which the GT signal is measured or generated is configured as an input signal, and the GT signal is shifted such that a batch window having a size W includes spikes of the GT signal to configure first training data. In this case, the GT signal of the first training data may be configured by shifting the center of the batch window regularly or randomly from −W/2 to W/2 from the time position of a minimum peak of the spikes.

Experimental results of a learning model including a neural network model that restores high-sampled and/or high-frequency biosignals from low-sampled and/or low-frequency biosignals will be described with reference to FIGS. 9 to 10.

FIG. 9 shows a high-frequency biosignal 920 restored by a learning model that has learned a low-sampled and low-frequency input biosignal 910 ten times, a high-frequency biosignal 930 restored by the learning model that has learned the low-sampled and low-frequency input biosignal 910 100 times, a high-frequency biosignal 940 restored by the learning model that has learned the low-sampled and low-frequency input biosignal 910 200 times, and a GT signal 950.

As can be ascertained from FIG. 9, the neural network model according to the embodiment of the present disclosure restores a biosignal almost identical to the GT signal in the case of learning the input biosignal more than 100 times.

FIG. 10A shows results obtained by enlarging a spike signal obtained by inputting a low-sampled and low-frequency biosignal to the first neural network model and then inputting the output of the first neural network model to the second neural network model 740 that improves a sampling frequency. FIG. 10B shows results obtained by upsampling the low-sampled and low-frequency biosignal and then inputting the biosignal to the neural network model. Each neural network mode was trained individually according to an upsampling rate.

It can be ascertained from FIGS. 10A and 10B that high-sampled biosignals of 8 times or more are similar to a GT signal in the case of the neural network model of FIG. 10A, and most of outputs are similar to the GT signal in the case of the neural network model of FIG. 10B.

The present disclosure described above can be implemented as computer-readable code in a medium on which a program is recorded. Computer-readable media include all types of recording devices in which data that can be read by a computer system is stored. Examples of computer-readable media include a hard disk drive (HDD), a solid state drive (SSD), a silicon disk drive (SDD), a ROM, a RAM, a CD-ROM, a magnetic tape, a floppy disk, an optical data storage device, etc. Further, the computer may include a processor of each device.

Meanwhile, the program may be specially designed and configured for the present disclosure, or may be known to those skilled in the art in the field of computer software. Examples of the program may include not only machine language code generated by a compiler but also high-level language code that can be executed by a computer using an interpreter or the like.

In the specification of the present disclosure (particularly in the claims), the use of the term “above” and similar indicating terms may correspond to both singular and plural. In addition, when a range is described in the present disclosure, describing each individual value constituting the range in the detailed description of the disclosure is equivalent to comprising a disclosure to which individual values belonging to the range are applied (unless otherwise stated).

Unless an order is explicitly stated for steps constituting the method according to the present disclosure, the steps may be performed in any suitable order. The present disclosure is not necessarily limited to the order of description of the steps. The use of all examples or exemplary terms (e.g., etc.) in the present disclosure is simply to explain the present disclosure in detail, and the scope of the present disclosure is limited due to the examples or exemplary terms unless limited by the claims. In addition, those skilled in the art can appreciate that various modifications, combinations, and changes can be made according to design conditions and factors within the scope of the appended claims or equivalents thereto.

Therefore, the spirit of the present disclosure should not be limited to the above-described embodiments, and not only the claims to be described later, but also all ranges equivalent to these claims are within the scope of the spirit of the present disclosure.

According to the apparatus and method for restoring high-frequency signals according to embodiments of the present disclosure, it is possible to obtain biosignals such as ECoG and spike-based brain signals without using an invasive biosignal measurement method, thereby preventing subject discomfort or risk due to surgery.

According to the apparatus and method for restoring high-frequency signals according to embodiments of the present disclosure, a restoration apparatus can restore high-sampled and high-frequency biosignals from low-sampled and low-frequency biosignals measured by a sensor device, thereby solving problems regarding power and storage spaces of wearable biosignal measurement devices.

What is claimed is:

1. A method of restoring a high-frequency biosignal, comprising:
loading a first biosignal by a processor; and
converting, by the processor, the first biosignal corresponding to a low-frequency signal into a second biosignal corresponding to a high-frequency signal on the basis of a first neural network model.

2. The method of claim 1, wherein the converting into the second biosignal comprises:
converting the first biosignal corresponding to a low-frequency signal into a third biosignal corresponding to a high-frequency signal on the basis of a neural network model; and
converting the third biosignal into the second biosignal having an improved sampling frequency on the basis of a second neural network model. 20

3. The method of claim 1, further comprising, prior to the converting into the second biosignal, improving a sampling frequency of the first biosignal corresponding to a low-frequency signal and converting the first biosignal into the third biosignal corresponding to a low-frequency signal,
wherein the converting into the second biosignal comprises converting the third biosignal corresponding to a low-frequency signal having an improved sampling frequency into the second biosignal corresponding to a high-frequency signal on the basis of the first neural network model.

4. The method of claim 1, wherein the first biosignal and the second biosignal include frequency band components of different types of biosignals.

5. The method of claim 4, wherein the first biosignal and the second biosignal include frequency band components of different types of biosignals obtained from different parts of a human body.

6. The method of claim 1, wherein the first biosignal is a non-invasively measured biosignal, and the second biosignal includes a frequency band component of an invasively measured biosignal.

7. The method of claim 1, wherein the first biosignal is a signal based on a motion measured by a sensor attached to a person corresponding to a subject, and
the converting into the second biosignal comprises converting the first biosignal into an electromyography (EMG) signal corresponding to the second biosignal on the basis of the first neural network model.

8. The method of claim 1, wherein the first neural network model is a machine learning-based learning model trained on the basis of training data in which input signals corresponding to low-frequency signals are labeled with ground truth (GT) signals corresponding to high-frequency signals, and
first input signals corresponding to low-frequency signals are labeled on first GT signals corresponding to high-frequency signals in a predetermined ratio of first training data in each mini-batch of the training data, and each of the first GT signals includes at least one spike.

9. An apparatus for restoring a high-frequency biosignal, comprising:
a processor; and
a memory electrically connected to the processor and storing at least one code executed by the processor,
wherein the memory stores code for causing, when executed by the processor, the processor to convert a first biosignal corresponding to a low-frequency signal into a second biosignal corresponding to a high-frequency signal on the basis of a first neural network model.

10. The apparatus of claim 9, wherein the memory further stores code for causing the processor to convert the first biosignal corresponding to a low-frequency signal into a third biosignal corresponding to a high-frequency on the basis of a first neural network model and to convert the third biosignal into the second biosignal having an improved sampling frequency on the basis of a second neural network model.

11. The apparatus of claim 9, wherein the memory further stores code for causing the processor to, prior to converting into the second biosignal, improve a sampling frequency of the first biosignal corresponding to a low-frequency signal, to convert the first biosignal into the third biosignal corresponding to a low-frequency signal, and to convert the third biosignal corresponding to a low-frequency signal having an improved sampling frequency into the second biosignal corresponding to a high-frequency signal on the basis of the first neural network model.

12. The apparatus of claim 9, wherein the first biosignal and the second biosignal include frequency band components of different types of biosignals.

13. The apparatus of claim 12, wherein the first biosignal and the second biosignal include frequency band components of different types of biosignals obtained from different parts of a human body.

14. The apparatus of claim 9, wherein the first biosignal is a non-invasively measured biosignal, and the second biosignal includes a frequency band component of an invasively measured biosignal.

15. The apparatus of claim 9, wherein the first biosignal is a signal based on a motion measured by a sensor attached to a person corresponding to a subject, and
the memory further stores code for causing the processor to convert the first biosignal into an electromyography (EMG) signal corresponding to the second biosignal on the basis of the first neural network model.

16. The apparatus of claim 9, wherein the first neural network model is a machine learning-based learning model trained on the basis of training data in which input signals corresponding to low-frequency signals are labeled with ground truth (GT) signals corresponding to high-frequency signals, and
first input signals corresponding to low-frequency signals are labeled on first GT signals corresponding to high-frequency signals in a predetermined ratio of first training data in each mini-batch of the training data, and each of the first GT signals includes at least one spike.
